(19) United States
(12) Patent Application Publication
(10) Pub. No.: US 2016/0007997 A1
(43) Pub. Date: Jan. 14, 2016

(54) Bridge vessels-proximal anastomosis supporting device for coronary artery bypass grafting and manufacturing method thereof

(71) Applicants: Chengxiong GU, Taipei (TW); Lei LI, Taipei (TW); Yang Yu, Taipei (TW)

(72) Inventors: Chengxiong GU, Taipei (TW); Lei LI, Taipei (TW); Yang Yu, Taipei (TW)

(21) Appl. No.: 14/331,207

(22) Filed: Jul. 14, 2014

(51) Int. Cl. A61B 17/11; A61M 1/36

(52) U.S. Cl. CPC A61B 17/11; A61M 1/3666; A61B 2017/1107; A61B 2017/1132

(57) ABSTRACT

A bridge vessels-proximal anastomosis supporting device for coronary artery bypass grafting and a manufacturing method thereof. The bridge vessels-proximal anastomosis supporting device for the coronary artery bypass grafting comprises an arc-shaped body, an elliptical bottom edge is arranged at the lower part of the arc-shaped body, a circular port is arranged at the right side of the arc-shaped body, a notch is formed between the rightmost end of the bottom edge and the lowermost end of the port, the diameter R of the circular port is 4 mm, 5 mm, 6 mm, 7 mm or 8 mm, the major axis a of the elliptical bottom edge is 13 mm, and the minor axis of the elliptical bottom edge is the same as R. The arc-shaped body is formed by weaving titanium-nickel alloy wires. The bottom edge and the port are provided with a supporting edge made of a heavy metal material, and the supporting edge is fixed with the wires through medical non-absorbable sutures in a wrapping manner.

### BACKGROUND OF INVENTION

1. Field of the Invention

The present invention relates to the field of medical and surgical supplies technologies, in particular to a bridge vessels-proximal anastomosis supporting device for coronary artery bypass grafting and a manufacturing method thereof

2. Description of Related Art

Coronary artery bypass graft surgery is the treatment of coronary artery disease book disease commonly surgery. The surgery used from the chest, leg or arm removed within a vein or artery, proximal anastomosis in the ascending aorta, across the stenosis or obstruction distal coronary arteries to improve blood supply to the heart and reduce the probability of occurrence of myocardial infarction to improve the quality of life of patients and prolong life. Due to coronary artery bypass graft proximal anastomosis is a slope, direct suture in the ascending aorta, the conventional bypass surgery will collapse and postoperative anastomotic bleeding blood clots cemented oppression anastomosis situation, resulting in graft stenosis or occlusion, reach the effect of improving myocardial blood flow, after long-term patency rate also decreased, there will be severe proximal anastomotic thrombosis, endangering the lives of patients. Currently, patients have yet to find the appropriate methods of prevention.

### SUMMARY OF THE INVENTION

The present invention is to provide a coronary artery bypass graft proximal anastomosis of graft support means, which has coronary artery bypass grafting avoid appearing vascular anastomosis collapse or extrusion, and thereby reduce surgical risk characteristics. Further, it is possible to prevent graft contracture caused by occlusion of the proximal anastomosis, and thereby improve the long-term postoperative myocardial blood flow rate characteristics. Further, and it has an easy-vitro study of anastomotic location features. Finally, the present invention also provides a method for preparing the support means.

To achieve the above object, the present invention provides a bridge vessels-proximal anastomosis supporting device for coronary artery bypass grafting comprises an arc-shaped body. An elliptical bottom edge is arranged at the lower part of the arc-shaped body, a circular port is arranged at the right side of the arc-shaped body, a notch is formed between the rightmost end of the bottom edge and the lowermost end of the port, the diameter R of the circular port is 4 mm, 5 mm, 6 mm, 7 mm or 8 mm, the major axis a of the elliptical bottom edge is 13 mm, and the minor axis of the elliptical bottom edge is the same as R.

Said arc-shaped body is formed by weaving titanium-nickel alloy wires, the wire having an outer diameter of 0.15 mm, and the wires in the same direction are parallel to each other and wires in different directions are distinguished by the diamond-shaped space in between, while the adjacent wire the distance between the axis of timber D is: D=2 mm.

The bottom edge and the port are provided with a supporting edge made from heavy metal material. The supporting edge is fixed with the wires through medical non-absorbable sutures in a wrapping manner onto the bottom edge and the port.

The cross-section of the support edge is circular, and has an outer diameter of 0.3 mm.

The support edge is made of titanium.

One method for manufacturing a bridge vessels-proximal anastomosis supporting device for coronary artery bypass grafting comprising the following steps:

a) making the arc-shaped body: take 0.15 mm diameter of nitinol wire, and according to the size of arc-shaped body cross-woven the wire into a diamond mesh, and then processed by heat molding process;

b) making the support edge of the bottom edge and the port: take titanium wire in diameter of 0.3 mm and form into an ellipse or circular shape that matches with the shape of the bottom edge and the port; and

c) fix the supporting edge with the wires through medical non-absorbable sutures in a wrapping manner onto the bottom edge and the port.

Said supporting edge is made from silver, with a silver purity of not less than 99.9%.

Another method for manufacturing a bridge vessels-proximal anastomosis supporting device for coronary artery bypass grafting comprising the following steps:

a) making the arc-shaped body: take 0.15 mm diameter of nitinol wire, and according to the size of arc-shaped body cross-woven the wire into a diamond mesh, and then processed by heat molding process;

b) making the support edge of the bottom edge and the port: take silver wire in diameter of 0.3 mm with a purity of not less than 99.9%, and form into an ellipse or circular shape that matches with the shape of the bottom edge and the port; and

c) fix the supporting edge with the wires through medical non-absorbable sutures in a wrapping manner onto the bottom edge and the port.

The present invention has the following advantages: 1. play a supporting intraoperative vascular anastomosis does not appear in the case of collapse or extrusion. The present invention is coronary artery bypass graft proximal anastomosis of graft main arc-shaped body supporting means are respectively provided at both ends of the oval and round bottom of a port having a different diameter (according to the invention with all dimensions except adjacent wire axis distance away, the rest are part of the outer edge of the dimensions). Thus, the support device can be fixed to the inside of anastomosis, vascular anastomosis that plays a supporting role. After optimization, the support means having a memory function by woven wire, so that the supporting device is deformed by the force, even, after removal of the external force, the support device can automatically restore the original shape in time, so as to effectively prevent an external force anastomosis under the action of the collapse. 2. after delaying anastomosis occlusion, in order to maintain long-term graft patency and improve long-term postoperative patency rates. Ministry anastomotic vascular occlusion is mainly due to graft contracture caused.

The support device of the present invention to take a good shape memory nitinol wire braid. The alloy after heat treatment after forming process, the shape memory capabilities further enhanced. That is, the supporting device not only has a certain strength, can overcome vasospasm and thus prevent occlusion of the anastomosis; and excellent toughness in even deformed by external pressure, can still be larger in the external pressure becomes smaller when the immediately restore the original shape. Thus, effectively preventing graft contracture impact. 3. easy for patients to quickly and accurately complete reexamination after coronary angiography. The supporting device has a support edge for the bottom edge and port, the supporting edge is made of titanium or a purity of not less than 99.9% of silver produced by the edge and the support of medical non-absorbable sutures wire fixed on the package. This not only supports the edge firmly fixed, and titanium or silver material can block X-rays pass. This is equivalent to that provided support device detectability mark, according to the marker to find the exact location of the anastomosis. 4. good ventilation, easy to use. The arc-shaped body is made of the support means weave, has good air permeability, no support means and the vessel wall there adhesion situation. The surgical mesh arc-shaped body conducive to the adoption of a fixed wire, and the support means precise size, shape science, and blood vessels can achieve a good fit, use, simply through the wire mesh 2 to 3 pin fixation can be very convenient. 5. the preparation process is reasonable. The preparation process of the support means comprises thermoforming step, which can enhance the shape memory wire. 6 and adaptable. The main arc-shaped body has a notch, so that when used in concrete, the diameter of the main arc-shaped body has a certain expansion margin, the supporting device can adapt to a wide range of the inner diameter of the individual vessels.

### BRIEF DESCRIPTION OF THE DRAWINGS

Below with the accompanying drawings and embodiments of the present invention is further described:

FIG. 1 is a schematic perspective view of a preferred embodiment of present invention;

FIG. 2 is a front view of an embodiment of a schematic structural view;

FIG. 3 is a right side view of FIG. 2;

FIG. 4 is a bottom view of FIG. 3; and

FIG. 5 is an enlarged view of portion A 2.

### DETAILED DESCRIPTION OF THE INVENTION

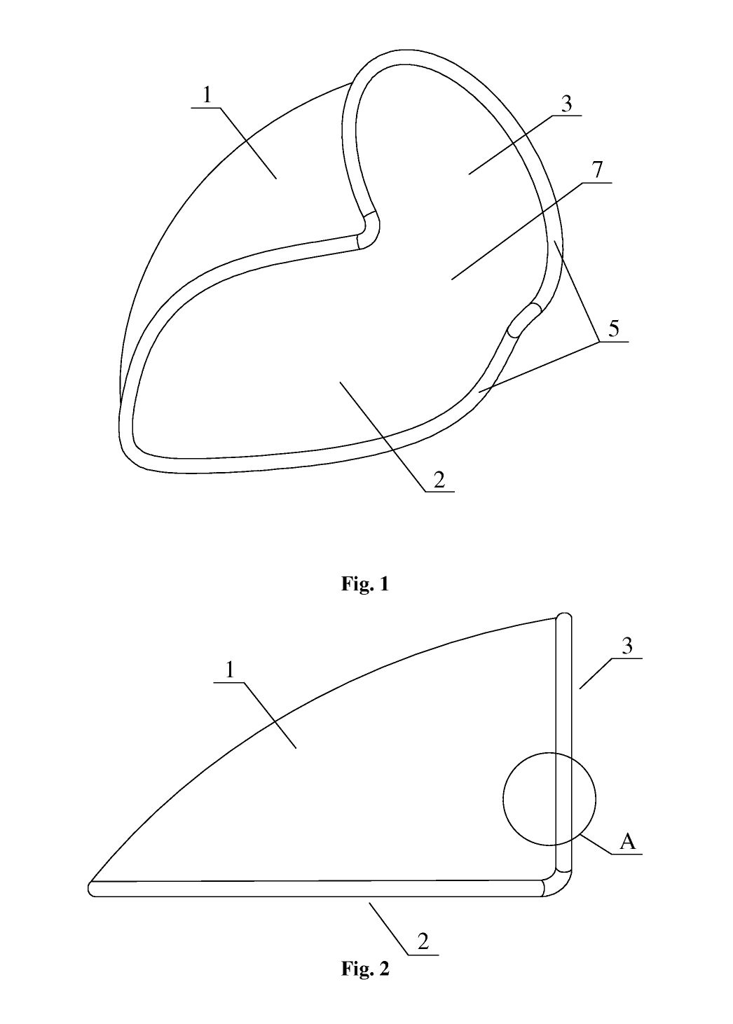

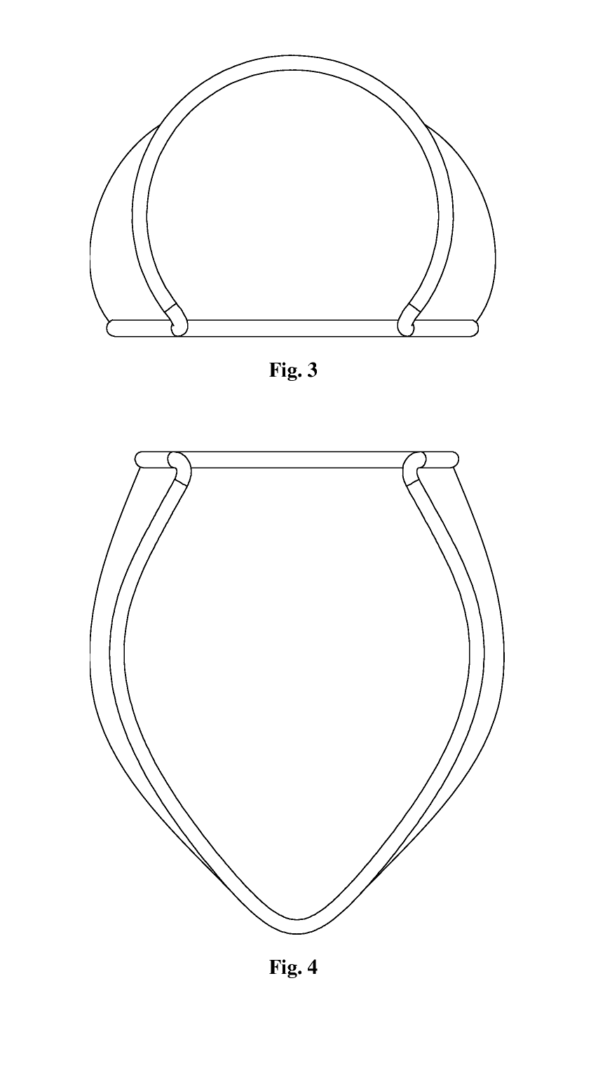

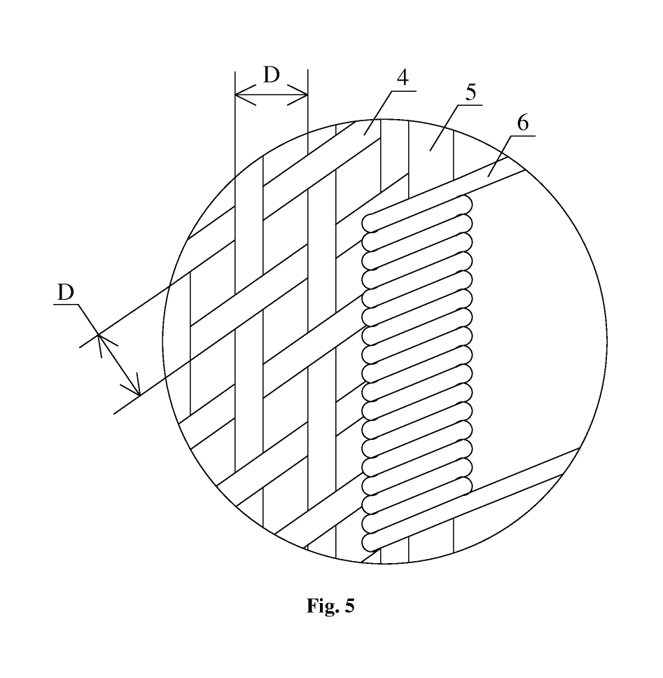

The following description is only preferred embodiments of the present invention thus does not limit the scope of the invention. Embodiment, as shown in FIG. 1 to FIG. 4: bridge vessels-proximal anastomosis supporting device for coronary artery bypass grafting comprises an arc-shaped body (1), an elliptical bottom edge (2) is arranged at the lower part of the arc-shaped body (1), a circular port (3) is arranged at the right side of the arc-shaped body (1), a notch (7) is formed between the rightmost end of the bottom edge (2) and the lowermost end of the port, the diameter R of the circular port (3) is 4 mm, 5 mm, 6 mm, 7 mm or 8 mm, the major axis a of the elliptical bottom edge (2) is 13 mm, and the minor axis of the elliptical bottom edge (2) is the same as R. Such a circular diameter of different sizes and different preferred diameter of the blood vessel anastomosis. Bottom two elliptical axis a is: a=13 mm, the same as R minor axis. Notch 7 is located at the far right and bottom edge of the port 2 in the lower end 3, so that the notch 7 can make the supporting means having a diameter size of the expansion margin. Arc-shaped body 1 is curved, so that the supporting means can move towards a better and vascular anastomosis and to ensure the support device and the vessel wall having a similar surface tension.

FIG. 5: the arc-shaped body 1 is made from cross woven wire 4. The outer diameter of wire 4 is 0.15 mm, and the wires 4 in the same direction are parallel to each other, and wires 4 in different directions have a diamond-shaped spacing in between. Meanwhile, the adjacent wire 4 has an axis distance D of: D=2 mm. To provide the arc-shaped body 1 with a good shape memory, nickel-titanium alloy is used for wire 4. In order to postoperative irradiation was observed by X-ray, can clearly find the location of vascular anastomosis, bottom edge 2 and port 3 is provided with a support edge 5 made of heavy material. The supporting edge 5 is fixed with the wires 4 through medical non-absorbable sutures in a wrapping manner onto the bottom edge 2 and the port 3. Of course, the heavy metal used in supporting edge 5 can be any common material that is un-harmful to human body. Thus, by X-ray irradiation, the support edge 5 of the X-rays through the barrier which can easily be observed, and the support edge 5 of the place where the location is the vascular anastomosis. For optimization the cross-section of supporting edge 5 is circular shape, and the circular shape has an outer diameter of 0.3 mm. For optimization, the support edge 5 may use heavy metal of silver or titanium. When using silver, silver purity is of not less than 99.9%.

Method for manufacturing a bridge vessels-proximal anastomosis supporting device for coronary artery bypass grafting comprising the following steps:

a) making the arc-shaped body: take 0.15 mm diameter of nitinol wire, and according to the size of arc-shaped body cross-woven the wire into a diamond mesh, and then processed by heat molding process;

b) making the support edge of the bottom edge and the port: take silver wire in diameter of 0.3 mm with a purity of not less than 99.9%, and form into an ellipse or circular shape that matches with the shape of the bottom edge and the port; and

c) fix the supporting edge with the wires through medical non-absorbable sutures in a wrapping manner onto the bottom edge and the port.

The application method of the present invention is: a) selected and vascular considerable size of the device, b) with 4-stich the bottom edge 2 is fixed to anastomosis around, c) check the firmness of the anastomosis: uses tweezers to gently pull the supporting device to ensure no displacement of the supporting device, d) with 2 to 3-stich to fix bridge vessels with the wire 4 of port 3.

Nitinol thermoforming process belong to existing technologies, thus will not repeat them here.

1. A bridge vessels-proximal anastomosis supporting device for coronary artery bypass grafting comprises an arc-shaped body; an elliptical bottom edge is arranged at the lower part of the arc-shaped body, a circular port is arranged at the right side of the arc-shaped body, and a notch is formed between the rightmost end of the bottom edge and the lowermost end of the port, the diameter R of the circular port is 4 mm, 5 mm, 6 mm, 7 mm or 8 mm, the major axis a of the elliptical bottom edge is 13 mm, and the minor axis of the elliptical bottom edge is the same as R.

2. The bridge vessels-proximal anastomosis supporting device for coronary artery bypass grafting as claimed in claim 1, wherein it is characterized in that: said arc-shaped body is formed by weaving titanium-nickel alloy wires; said wire has an outer diameter of 0.15 mm, and the alloy wires in the same direction are parallel to each other and wires in different directions are distinguished by diamond-shaped spacing, while the adjacent wires have an axial distance D of: D=2 mm.

3. The bridge vessels-proximal anastomosis supporting device for coronary artery bypass grafting as claimed in claim 2, wherein it is characterized in that: said bottom edge and the port are provided with a supporting edge made from heavy metal material, said supporting edge is fixed with the wires through medical non-absorbable sutures in a wrapping manner onto the bottom edge and the port.

4. The bridge vessels-proximal anastomosis supporting device for coronary artery bypass grafting as claimed in claim 3, wherein it is characterized in that: the cross-section of said supporting edge is circular shape, said circle shaped has an outer diameter of 0.3 mm.

5. The bridge vessels-proximal anastomosis supporting device for coronary artery bypass grafting as claimed in claim 4, wherein said supporting edge is made of titanium.

6. The bridge vessels-proximal anastomosis supporting device for coronary artery bypass grafting as claimed in claim 5, wherein the method for making said coronary artery bypass graft proximal anastomosis support device comprising the following steps:
a) making the arc-shaped body: take 0.15 mm diameter of nitinol wire, and according to the size of arc-shaped body cross-woven the wire into a diamond mesh, and then processed by heat molding process;
b) making the support edge of the bottom edge and the port: take titanium wire in diameter of 0.3 mm and form into an ellipse or circular shape that matches with the shape of the bottom edge and the port; and
c) fix the supporting edge with the wires through medical non-absorbable sutures in a wrapping manner onto the bottom edge and the port.

7. The bridge vessels-proximal anastomosis supporting device for coronary artery bypass grafting as claimed in claim 4, wherein it is characterized in that: said supporting edge is prepared from silver with a purity of not less than 99.9%.

8. The bridge vessels-proximal anastomosis supporting device for coronary artery bypass grafting as claimed in claim 7, wherein the method for making said coronary artery bypass graft proximal anastomosis support device comprising the following steps:
a) making the arc-shaped body: take 0.15 mm diameter of nitinol wire, and according to the size of arc-shaped body cross-woven the wire into a diamond mesh, and then processed by heat molding process;
b) making the support edge of the bottom edge and the port: take silver wire in diameter of 0.3 mm with a purity of not less than 99.9%, and form into an ellipse or circular shape that matches with the shape of the bottom edge and the port; and
c) fix the supporting edge with the wires through medical non-absorbable sutures in a wrapping manner onto the bottom edge and the port.
